(19) United States
(12) Patent Application Publication
(10) Pub. No.: US 2010/0216749 A1
(43) Pub. Date: Aug. 26, 2010

(54) Combination Therapy for Neuroprotection

(75) Inventors: Sung-Hwa Yoon, Suwon-si (KR); Ja-Kyeong Lee, Seoul (KR); Ho-Joon Park, Suwon-si (KR); Young-Gyun Shin, Suwon-si (KR); Hae-Un Lee, Suwon-si (KR); Seung-Woo Kim, Incheon (KR); Hyun-Ji Kim, Suwon-si (KR)

Correspondence Address: THE WEBB LAW FIRM, P.C., 700 KOPPERS BUILDING, 436 SEVENTH AVENUE, PITTSBURGH, PA, 15219, US

(73) Assignee: S.H. PHARMA., LTD, Seoul (KR)

(21) Appl. No.: 12/681,680

(22) PCT Filed: Oct. 3, 2008
(86) PCT No.: PCT/KR08/05841

§ 371 Date: Apr. 5, 2010

(30) Foreign Application Priority Data
Oct. 5, 2007 (KR) 10-2007-0100351
Oct. 2, 2008 (KR) 10-2008-0097093

(51) Int. Cl. A61K 31/616; A61K 31/138; A61P 25/00

(52) U.S. Cl. 514/164; 514/651

(57) ABSTRACT

The present invention relates to combination therapy of a pyruvoyl derivative and an anti-oxidant, which is characterized by significant increase of the activity of microglia, inhibition of brain tissue damage by the activation of inflammatory cytokines, improvement of motor skill and recovery effect of neuronal damage. Compared to single treatment with each, this present invention provides a continuous high neuroprotective effect even after 6 hours from the onset of damage.

### TECHNICAL FIELD

The present invention relates to combination therapy for neuroprotection, more precisely a pharmaceutical composition for maximizing the effect of inhibiting cerebral infarction after cerebral ischemia and improving neurologic damage and motor ability by co-treatment with a pyruvoyl analogue and an anti-oxidant.

### BACKGROUND ART

Stroke is a major cerebrovascular disease, which is the leading cause of death as a single disease in Korea. Neurologic damage after cerebral ischemia is resulted from diverse in vivo processes as follows; intra/extra-cellular calcium balance is broken by continuous excitation of glutamate receptor (NMDA or non-NMDA receptor) by excessive secretion of excitatory amino acid neurotransmitter of central nervous system to induce neurotoxicity (Lipton et al., 1999); cell damage is caused by reactive oxygen species such as NO and O₂⁻ overproduced during reperfusion; and diverse reactions induced in mitochondria are also responsible for the neurologic damage. As ischemia-reperfusion progresses in central nervous system, acute neuronal death is caused by excitotoxicity, leading to delayed neuronal damage slowly progressed over hours to days (Kirino et al., 2000). Delayed neuronal death is secondary brain tissue damage progressed by neuroinflammation, which accompanies unexpected gene expressions over the time. It is possible to reduce irreversible cell damage if it is early diagnosed and treated properly.

Therapeutic agents clinically used for stroke are exemplified by fibrinolytic agents such as tissue plasminogen activator (referred as ‘tPA’ hereinafter) and urokinase; antiplatelet agents; vasodilators, calcium ion channel I blockers, etc. However, there are disadvantages in using these drugs, which are these drugs have to be administered within 3 hours from onset and they accompany side effects including non-specific bleeding, fibrinogen lysis, etc (Hacke et al 1995, Clark et al., 2000)

Pyruvate is generated by pyruvate kinase in the last phase of glycolysis and sometimes generated in other metabolic pathways such as aminotransfer process, etc. It has been recently reported that pyruvate not only plays an important role as a metabolic intermediate but also has anti-oxidative activity and function of eliminating free radicals. Functions of pyruvate disclosed so far are as follows: (1) playing a role as an intermediate of TCA cycle and as a metabolic substance, (2) eliminating oxygen peroxide by the reaction process of CH₃CCOOO⁻+H₂O₂→CH₃COO⁻+H₂O+CO₂, (Holleman, 1904) (3) eliminating hydroxyl radical (OH), one of reactive oxygen species, (Varma et al., 1998) and (4) inotropic activity and activating sarcoplasmic reticular ATPase. Cell protective function of pyruvate has been studied under various disease conditions. For example, protective effect of pyruvate was first confirmed in acute kidney disease induced by hydrogen peroxide when pyruvate chloride was administered by intravenous injection (Salahudeen et al., 1991). Since then, protective effect of pyruvate against ischemia related stress has been further reported in myocardium, intestine and liver. The protective effect in animal models having cataract induced by galactose or diabetes, cerebral ischemia and concealed hemorrhage was also reported. However, pyruvate is significantly limited in practical use as a therapeutic agent because (1) it has very low solubility and very unstable in aqueous solution (Montgomery et al., 1956), and (2) it fundamentally becomes a strong inhibitor of TCA cycle by being converted into parapyruvate (Fink, 2003)

To overcome the above limits of pyruvate, various pyruvate derivatives, for example alkylpyruvate represented by formula A, have been studied:

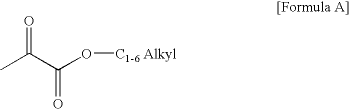

One of the pyruvate derivatives, ethylpyruvate (referred as ‘EP’ hereinafter) has advantages as a potential efficient and powerful substitute for pyruvate as follows: first, it is an ester analogue so that it has high lipophilicity and high cytopermeability second, it has low solubility in saline or water, but it demonstrates significantly increased solubility in calcium solution (Ringer solution); third, when it is dissolved in calcium solution (Ringer solution), it is stabilized by being dimer to form anionic enolate, so that it can play a role as a pyruvate precursor; and fourth, it is a very safe substance, so it is permitted as a food additive (Sims et al., 2001; Reade et al 2005).

The present inventors previously reported that EP showed excellent neuroprotective effect in a stroke animal model, demonstrating high potential as a therapeutic agent for stroke (Yu et al., 2005). And, the present inventors got a patent (Korean Patent No. 686,652) for a neuroprotective agent containing EP as an active ingredient. More specifically, when EP is administered into the abdominal cavity 12 hours after ischemia-reperfusion, the size of infarction can be reduced to less than 50%. When EP is administered as late as 24 hours after ischemia-reperfusion, the size of infarction can be approximately 20% reduced (Yu et al., 2005). Rota-rod test confirmed that infarction suppressing effect was accompanied by motor function recovery effect. During this process, anti-inflammatory effect of EP such as inhibiting the activation of microglia and inhibiting the expression of pro-inflammatory cytokines were also observed (Yu et al., 2005). Anti-oxidative effect of EP was confirmed using primary culture cells (Kim et al 2005). Particularly, post-treatment effect of EP was greater than any other candidate drugs reported so far. EP is an intracellular natural material, which is also a great advantage. Considering high cytopermeability and safety along with diverse functions of EP, it is expected to be an efficient therapeutic agent for such disease developed by complicated processes as stroke.

Acetylsalicylic acid (product name: aspirin) represented by formula B is one of drugs proved to have preventive and therapeutic effect on cardiovascular disease:

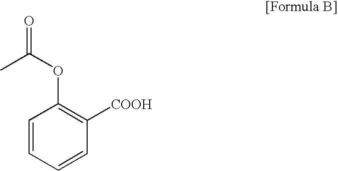

Acetylsalicylic acid inhibits blood vessel blocking by suppressing irreversible platelet aggregation. That is, acetylsalicylic acid replaces cyclooxygenase-1 (referred as ‘COX-1’ hereinafter) and cyclooxygenase-2 (referred as ‘COX-2’ hereinafter) which are essential enzymes for thromboxane A₂ (referred as ‘TXA₂’ hereinafter) formation that is a strong platelet aggregation inducer and at the same time a vasoconstrictor with acetyl group, resulting in the inhibition of platelet aggregation at last (Vane et al., 1971). As a result, it reduces the size of emboli and inhibits vasoconstriction in ischemia progressed central nervous system. It has also been reported that acetylsalicylic acid has anti-inflammatory effect by suppressing NF-kB and nervous system protective effect by anti-oxidative activity (Kopp et al., 1994). It is also known that acetylsalicylic acid inhibits damage caused by hypoxia by prolonging intracellular ATP loss (De Cristobal et al., 2002). To inhibit TXA₂, a low dose of acetylsalicylic acid (≧30 mg/day) is enough. But, to induce neuroprotective activity by anti-inflammatory or anti-oxidative functions, a higher dose (3˜6 g/day) is necessary. According to the result of meta-analysis examining the effect of aspirin on cardiovascular disease, if 75˜325 mg of aspirin is administered daily basis, it brings preventive and therapeutic effect on cardiovascular disease in the long term.

5-Aminosalicylic acid derivative represented by formula C is known to have strong anti-oxidative effect and preventive (McKenzie et al., 1999) and therapeutic effect on acute or degenerative nervous disease by its anti-oxidative and anti-inflammatory functions (US Patent Publication No. 2005-239896, Korean Patent No. 751,888 and Korean Patent no. 668,111):

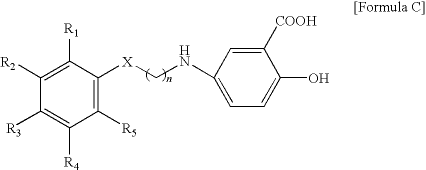

Wherein,

R₁-R₅ are independently H, C₁₋₄ alkyl, halogen, hydroxy, C₁₋₄ alkoxy, CF₃ or nitro,

n is an integer of 0-4,

X is CH₂, O, S or SO.

Fluoxetine (chemical name: N-methyl-[4-(trifluoromethyl)phenoxy]benzenepropaneamine) represented by formula D was developed by Eli Lilly and Company (U.S. Pat. No. 4,018,895) and approved by FDA (Food and Drug Administration, USA) in 1987. It has been the most prescribed antidepressant world-widely since then:

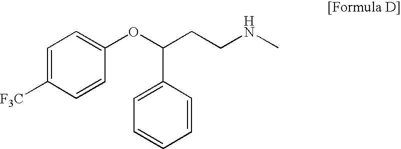

Fluoxetine increases serotonin, one of neurotransmitters regulating human emotion in brain (Jolkkonen et al., 2000). Fluoxetine significantly reduces anticholinergic side effects generally carried by the conventional antidepressant such as insomnia, weight gaining, visual disturbance, cardiac arrhythmia, xerostomia and constipation. In addition, fluoxetine has an advantage of convenience of administration that is once a day any time with or without food and with another medicine. Fluoxetine has been used for the treatment of post-stroke depression (PSD) observed in most stroke patients and it has demonstrated motor function and cognition recovery effect along with the alleviation of depression (Wiart et al., 2000). Fluoxetine is a safe substance that can treat obsessive-compulsive disorder, addephagia, anthropophobia, cleptomania, post-traumatic stress disorder developed after natural disaster, etc, and panic disorder showing seizure Generation of reactive oxygen species or nitrogen oxide harmful for nerve cells induces neuronal death, which is involved in various nervous system diseases such as ischemic stroke and traumatic head injury. (Lim et al., 2008, Horsfield et al., 2000). According to the previous reports, fluoxetine shows diverse neuroprotective effects.

It might be necessary to co-treat one or more drugs together to treat a disease developed by diverse mechanisms such as stroke. At the same time, it is also important to develop a novel drug for post-treatment that can bring effect when it is treated even after a while from the onset of a disease. However, the preventive and treatment effects on stroke along with neuroprotective effect of combination of anti-oxidants selected from the group consisting of alkylpyruvate represented by formula A, acetylsalicylic acid represented by formula B, 5-aminosalicylic acid derivative represented by formula C and fluoxetine represented by formula D has not been reported, yet.

### DISCLOSURE

### Technical Problem

The present inventors tried to find out a method maximizing the effect of inhibiting cerebral infarction after cerebral ischemia and improving motor ability and neurologic damage by combination of drugs demonstrating superior effect on neurologic damage caused by stroke. At the same time, the present inventors tried to find out a method which was still effective even when the treatment was performed a while after the onset of a disease. As a result, the present inventors confirmed that combination therapy of a pyruvoyl derivative and an anti-oxidant met the above goal, leading to the completion of this invention.

It is an object of the present invention to provide a neuroprotective agent containing a pyruvoyl derivative and an antioxidant as active ingredients.

It is another object of the present invention to provide a pharmaceutical kit for neuroprotection containing a pyruvoyl derivative and an antioxidant as active ingredients.

It is further an object of the present invention to provide a pharmaceutical package for neuroprotection containing a pyruvoyl derivative and an antioxidant.

### TECHNICAL SOLUTION

First, the present invention relates to a neuroprotective agent containing:

(1) pyruvoyl derivative represented by formula 1; and

(2) one or more antioxidants selected from the group consisting of acetylsalicylic acid derivative represented by formula 2,5-aminosalicylic acid derivative represented by formula 3, fluoxetine derivative represented by formula 4 and pharmaceutically acceptable salts thereof as active ingredients:

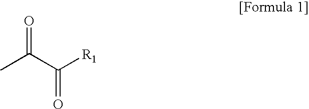

[Wherein, R₁ is (C1-C6) alkyl, (C6-C12) aryl, (C1-C6) alkoxy, (C6-C12) aryloxy, (C6-C12) ar (C1-C6) alkyl, (C6-C12) ar (C1-C6) alkyloxy, NR₂₁R₂₂ or (C1-C6) alkylthio; R₂₁ and R₂₂ are independently H, (C1-C6) alkyl, (C6-C12) aryl, (C6-C12) ar (C1-C6) alkyl, (C6-C12) arylcarbonyl or (C1-C6) alkylcarbonyl; Alkyl, aryl, alkoxy, aryloxy, aralkyl, aralkyloxy, arylcarbonyl and alkylcarbonyl of R₁, R₂₁ and R₂₂ can be substituted with one or more substituents selected from the group consisting of halogen, halo (C1-C6) alkyl (in particular, CF₃), halo (C1-C6) alkyl (C6-C12) aryloxy and hydroxy.]

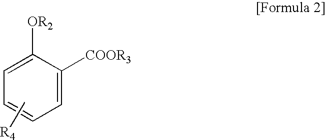

[Wherein, R₂ is H, (C1-C6) alkylcarbonyl or (C6-C12) arylcarbonyl; Arylcarbonyl of R₂ can be substituted with one or more substituents selected from the group consisting of (C1-C6) alkyl, hydroxy and halo (C1-C6) alkyl (in particular, CF₃); R₃ is H, (C1-C6) alkyl or (C6-C12) aryl; R₄ is H, (C1-C6) alkyl, halo (C1-C6) alkyl (in particular, CF₃), halogen, (C1-C6) alkoxy, hydroxy or nitro.]

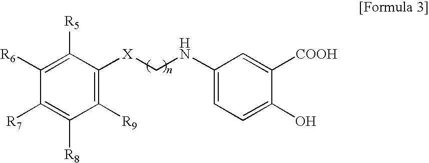

[Wherein, R₅-R₉ are independently H, (C1-C6) alkyl, halogen, hydroxy, (C1-C6) alkoxy, CF₃ or nitro; n is an integer of 0-4; X is CH₂, O, S or SO.]

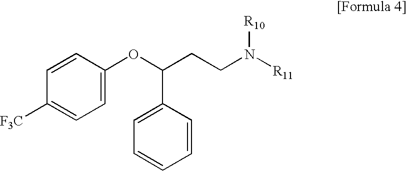

[Wherein, R₁₀ and R₁₁ are independently H, (C1-C6) alkyl or (C6-C12) aryl.]

Second, the present invention relates to a pharmaceutical kit for neuroprotection containing:

(1) pyruvoyl derivative represented by formula 1; and

(2) one or more antioxidants selected from the group consisting of acetylsalicylic acid derivative represented by formula 2,5-aminosalicylic acid derivative represented by formula 3, fluoxetine derivative represented by formula 4 and pharmaceutically acceptable salts thereof as active ingredients.

Third, the present invention relates to a pharmaceutical package for neuroprotection containing:

(1) pyruvoyl derivative represented by formula 1; and

(2) one or more antioxidants selected from the group consisting of acetylsalicylic acid derivative represented by formula 2,5-aminosalicylic acid derivative represented by formula 3, fluoxetine derivative represented by formula 4 and pharmaceutically acceptable salts thereof as active ingredients.

Hereinafter, the present invention is described in detail.

According to the present invention, combination therapy of a pyruvoyl derivative and an antioxidant can increase the treatment effect significantly, compared with single treatment, by inhibiting the activation of microglia and the activation of inflammatory cytokines to inhibit brain tissue injury. The present invention also provides a method demonstrating a prominent treatment effect for 6-24 hours after the onset of a disease, which gives longer therapeutic window than using a single treatment of either a pyruvoyl derivative or an antioxidant. Accordingly, the drugs of the present invention can be used for the treatment of various cerebrovascular diseases including cerebral ischemia, cerebral infarction, stroke, Huntington's disease, Lou Gehrig's disease or vascular dementia, and is particularly effective in preventing or treating stroke.

In this invention, the pyruvoyl derivative is selected from the group consisting of:

ethyl pyruvate (pyruvoyloxyethane);

propyl pyruvate;

isopropyl pyruvate;

n-butyl pyruvate;

sec-butyl pyruvate;

isobutyl pyruvate;

t-butyl pyruvate;

pentyl pyruvate;

hexyl pyruvate;

heptyl pyruvate;

octyl pyruvate;

phenyl pyruvate;

4-(4-trifluoromethylphenyloxy)phenyl pyruvate;

(4-trifluoromethyl)-2,3,5,6-tetrafluorophenyl pyruvate;

(4-trifluoromethyl)phenyl pyruvate;

(4-trifluoromethyl)benzyl pyruvate;

pyruvoylaminoethane;

pyruvoylaminomethane;

pyruvoylaminopropane;

pyruvoylaminobutane;

pyruvoylaminopentane;

pyruvoylaminobenzene;

pyruvoylamino(4-trifluoromethyl)benzene;

pyruvoylamino(4-trifluoromethylphenyl)methane;

pyruvoylamino(4-trifluoromethyl-2,3,5,6-tetrafluorophenyl)methane;

pyruvoylamino(4-trifluoromethyl-2,3,5,6-tetrafluoro)benzene; pyruvoylaminocarbonyl(4-trifluoromethyl)benzene;

pyruvoylamininediethane;

pyruvoylamininedibutane;

pyruvoylamininedihexane;

pyruvoylamininedioctane;

pyruvoyl(4-trifluoromethyl-2-hydroxyphenylcarbonyl)(ethyl)amine;

pyruvoyl(4-trifluoromethyl-2-hydroxyphenyl)(ethyl)amine;

pyruvoyl(4-trifluoromethylphenyl)(ethyl)amine;

pyruvoylthioethane; and

pyruvoylpropane.

The pharmaceutically acceptable salt of the present invention can include pharmaceutically acceptable acid addition salt, and solvates and hydrates thereof are included in the scope of the present invention. The pharmaceutically acceptable acid addition salt can be obtained from inorganic acids such as hydrochloric acid, nitric acid, phosphoric acid, sulfuric acid, hydrobromic acid, hydriodic acid, nitrous acid or phosphorous acid and nontoxic organic acids such as aliphatic mono/dicarboxylate, phenyl-substitutedalkanoate, hydroxy alkanoate/alkanedioate, aromatic acids, and aliphatic/aromatic sulfonic acids. Particularly, the pharmaceutically acceptable salt is exemplified by sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutylate, caprate, heptanoate, propiolate, oxalate, malonate, succinate, suberate, sebacate, fumalate, malate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylensulfonate, phenylacetate, phenylpropionate, phenylbutylate, citrate, lactate, β-hydroxybutylate, glycolate, malate, tartlate, methanesulfonate, propanesulfonate, naphthalene-1-sulfonate, naphthalene-2-sulfonate or mandelate, and hydrochloride is more preferred.

The acetylsalicylic acid derivative is selected from the group consisting of:

acetylsalicylic acid;

2-(2-hydroxy-4-(trifluoromethyl)benzoyloxy)-4-(trifluoromethyl)benzoic acid; and

2-(2-(2-oxopropanoyloxy)-4-(trifluoromethyl)benzoyloxy)-4-(trifluoromethyl)benzoic acid.

The 5-aminosalicylic acid derivative is selected from the group consisting of:
2-hydroxy-5-(2,3,5,6-tetrafluoro-4-(trifluoromethyl)benzylamino)benzoic acid;
2-hydroxy-5-(4-(trifluoromethyl)phenylethylamino)benzoic acid;
5-(2-(4-chlorophenoxy)ethylamino)-2-hydroxybenzoic acid;
5-(2-(2,4-dichlorophenoxy)ethylamino)-2-hydroxybenzoic acid;
2-hydroxy-5-(2-(2,4,5-trichlorophenoxy)ethylamino)benzoic acid;
2-hydroxy-5-(2-phenylsulfonylethylamino)benzoic acid;
2-hydroxy-5-(2-(4-iodophenoxy)ethylamino)benzoic acid;
5-(2-(4-bromophenoxy)ethylamino)-2-hydroxybenzoic acid;
2-hydroxy-5-(3-phenoxypropylamino)benzoic acid;
5-(2-(2,6-dichloro-4-fluorophenoxy)ethylamino)-2-hydroxybenzoic acid;
5-(3-(4-fluorophenoxy)propylamino)-2-hydroxybenzoic acid;
5-(2-(2,6-dichloro-4-fluorophenoxy)ethylamino)-2-hydroxybenzoic acid;
5-(2-(4-chloro-2-methylphenoxy)ethylamino)-2-hydroxybenzoic acid;
2-hydroxy-5-(2-p-tolyloxyethylamino)benzoic acid;
2-hydroxy-5-(2-phenoxyethylamino) benzoic acid;
5-(2-(2,6-difluorophenoxy)ethylamino)-2-hydroxy benzoic acid; and
2-hydroxy-5-(2-(4-methoxyphenoxy)ethylamino)benzoic acid.

The fluoxetine derivative is selected from the group consisting of:

fluoxetine;

3-(4-(trifluoromethyl)phenoxy)-N-methyl-3-phenylpropane-1-amine;

3-(4-(trifluoromethyl)phenoxy)-N,N-dimethyl-3-phenylpropane-1-amine;

N-(3-(4-(trifluoromethyl)phenoxy)-3-phenylpropyl)-N-phenylbenzeneamine; and

N-(3-(4-(trifluoromethyl)phenoxy)-3-phenylpropyl)-N-phenylbenzeneamine.

In this invention, the pyruvoyl derivative and the anti-oxidant can be included in a single or separated unit dosage form or can be administered simultaneously or at regular intervals or stepwise. So, the above two active ingredients can be formulated, together or separately, for oral administration (for example, tablets, hard or soft capsules, granules, chewing tablets, pills, powders, elixirs, suspensions, emulsions and syrups) or for parenteral administration (for example aerosols, sachets, sterile injections and sterile powders) according to the purpose of administration by mixing with pharmaceutically acceptable carriers. In the case of that the active ingredients of the present invention are formulated for oral administration such as tablets, hard or soft capsules, granules, chewing tablets, pills, powders, elixirs, suspensions, emulsions and syrups, they can be mixed with a binding agent such as Arabia rubber, corn starch, microcrystalline cellulose or gelatin, an excipient such as dicalcium phosphate or lactose, a disintegrating agent such as alginic acid, corn starch or potato starch, a lubricant such as magnesium stearate, a sweetening agent such as sucrose or saccharin and a flavor such as peppermint, methyl salicylate or fruit aroma. If the unit dosage is a capsule, a liquid carrier such as polyethylene glycol or fatty oil can additionally included, in addition to the above ingredients. Solution or suspension type injections can be administered parenterally. For example, intravenous administration, intramuscular administration or intra-abdominal administration can be used. In general, injectable solution or suspension can be prepared by mixing effective dose of the active ingredients with a pharmaceutically acceptable liquid carrier such as water, salt water, water dextrose and its related sugar solutions, non-volatile oil, ethanol, glycerin, and glycols such as polyethylene glycol, propylene glycol. If necessary, a supplement such as an antimicrobial agent, a chelating agent, a buffering agent and a preserving agent can be additionally included. The pharmaceutically acceptable carrier herein can be any supplement that is pharmaceutically pure and non-toxic in fact and does not affect the active ingredients.

In this invention, the preferable dose of a pyruvoyl derivative is 1-60 mg/kg and the preferable dose of an anti-oxidant is 1-40 mg/kg. However, the effective dose can be regulated according to age, gender, diet, health condition, severity of disease, administration method and frequency and combination of drugs etc.

### ADVANTAGEOUS EFFECT

According to the present invention, combination therapy of a pyruvoyl derivative and diverse anti-oxidants brings the effect of inhibiting brain tissue damage and improving motor ability and recovery of neurologic damage based on the significantly increased activation of microglia and the significant inhibition of the activation of inflammatory cytokines, compared to single treatment of each. If an anti-oxidant is administered 6 hours after the damage, the effect is significantly reduced. If a pyruvoyl derivative is administered alone, the effect starts decreasing after 6 hours from the damage. However, when the two active ingredients are co-treated, prominent neuroprotective effect is observed even after 6 hours from the damage.

### DESCRIPTION OF DRAWINGS

The above and other objects, features and advantages of the present invention will become apparent from the following description of preferred embodiments given in conjunction with the accompanying drawings, in which:

FIG. 1 is a photograph illustrating the ischemia of brain sections stained with TTC of an animal model with local ischemia induced by middle cerebral artery occlusion (referred as ‘MLAO’ hereinafter) and treated with ethyl pyruvate (referred as ‘EP’ hereinafter);

FIG. 2 is a graph illustrating the ischemia of brain sections over the EP concentration;

FIG. 3 is a graph illustrating the volume of whole ischemia over the EP administration time;

FIG. 4 is a graph illustrating the result of Rota-rod test after the EP administration;

FIG. 5 is a graph illustrating the result of static reflex test and forelimb placing test after the EP administration;

FIG. 6 is a photograph illustrating the ischemia of brain sections stained with TTC of an animal model with local ischemia induced by MCAO and treated with EP and acetylsalicylic acid independently or together;

FIG. 7 is a graph illustrating the ischemia of brain sections over the concentrations of EP and acetylsalicylic acid;

FIG. 8 is a graph illustrating the volume of whole ischemia over the administration time of EP and acetylsalicylic acid;

FIG. 9 is a graph illustrating the result of Rota-rod test after the co-administration of EP and acetylsalicylic acid;

FIG. 10 is a graph illustrating the result of static reflex test and forelimb placing test after the co-administration of EP and acetylsalicylic acid;

FIG. 11 and FIG. 12 are graphs illustrating the volume of whole ischemia over the administration time of EP and chloro-2-hydroxy-5-(2,3,5,6-tetrafluoro-4-(trifluoromethyl)benzylamino)benzoate after MCAO;

FIG. 13 is a graph illustrating the result of static reflex test and forelimb placing test after the co-administration of EP and chloro-2-hydroxy-5-(2,3,5,6-tetrafluoro-4-(trifluoromethyl)benzylamino)benzoate;

FIG. 14 is a graph illustrating the ischemia of brain sections stained with TTC of an animal model with local ischemia induced by MCAO and treated with EP and chloro-2-hydroxy-5-(2,3,5,6-tetrafluoro-4-(trifluoromethyl)benzylamino)benzoate independently or together;

FIG. 15 is a graph illustrating the volume of whole ischemia over the administration time of EP and chloro-5-(2-(4-chlorophenoxy)ethylamino)-2-hydroxybenzoic acid after MCAO;

FIG. 16 is a graph illustrating the result of static reflex test and forelimb placing test after the co-administration of EP and chloro-5-(2-(4-chlorophenoxy)ethylamino)-2-hydroxybenzoic acid;

FIG. 17 is a graph illustrating the volume of whole ischemia over the administration time of EP and chloro-5-(2-(2,4-dichlorophenoxy)ethylamino)-2-hydroxybenzoic acid after MCAO;

FIG. 18 is a graph illustrating the result of static reflex test and forelimb placing test after the co-administration of EP and chloro-5-(2-(2,4-dichlorophenoxy)ethylamino)-2-hydroxybenzoic acid;

FIG. 19 is a graph illustrating the volume of whole ischemia over the administration time of EP and chloro-2-hydroxy-5-(2-(2,4,5-trichlorophenoxy)ethylamino)benzoic acid after MCAO;

FIG. 20 is a graph illustrating the result of static reflex test and forelimb placing test after the co-administration of EP and chloro-2-hydroxy-5-(2-(2,4,5-trichlorophenoxy)ethylamino)benzoic acid;

FIG. 21 is a graph illustrating the volume of whole ischemia over the administration time of EP and chloro-2-hydroxy-5-(2-(4-methoxyphenoxy)ethylamino)benzoic acid after MCAO;

FIG. 22 is a graph illustrating the result of static reflex test and forelimb placing test after the co-administration of EP and chloro-2-hydroxy-5-(2-(4-methoxyphenoxy)ethylamino)benzoic acid;

FIG. 23 is a set of photographs ischemia of brain sections stained with TTC after the administration of fluoxetine chloride after MCAO and a graph illustrating the ischemia of brain sections over the concentration of fluoxetine chloride;

FIG. 24 is a graph illustrating the volume of whole ischemia over the administration time of fluoxetine chloride;

FIG. 25 is a graph illustrating the result of static reflex test and forelimb placing test after the co-administration of EP and fluoxetine chloride;

FIG. 26 is a graph illustrating the ischemia of brain sections stained with TTC of an animal model with local ischemia induced by MCAO and co-treated with pyruvoylamininediethane and 2-acetoxy-4-(trifluoromethyl)benzoic acid (triflusal);

FIG. 27 is a graph illustrating the ischemia of brain sections stained with TTC of an animal model with local ischemia induced by MCAO and co-treated with pyruvoylamininediethane and 2-hydroxy-4-trifluoromethylbenzoic acid (HTB);

FIG. 28 is a graph illustrating the ischemia of brain sections stained with TTC of an animal model with local ischemia induced by MCAO and co-treated with pyruvoylaminoethane and 2-acetoxy-4-(trifluoromethyl)benzoic acid (triflusal);

FIG. 29 is a graph illustrating the ischemia of brain sections stained with TTC of an animal model with local ischemia induced by MCAO and co-treated with pyruvoylaminoethane and 2-hydroxy-4-trifluoromethylbenzoic acid (HTB).

### BEST MODE

Practical and presently preferred embodiments of the present invention are illustrative as shown in the following Examples.

However, it will be appreciated that those skilled in the art, on consideration of this disclosure, may make modifications and improvements within the spirit and scope of the present invention.

Manufacturing Example 1

### Preparation of Pyruvoylaminoethane

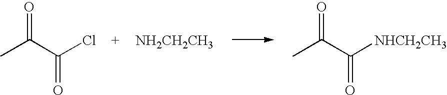

2 M Ethylamine (14.1 mL, 28.2 mmol) was dissolved in dichloromethane (40 mL). Triethylamine (4.0 mL, 28.2 mmol) was added thereto, followed by stirring for 5 minutes. Pyruvoyl chloride (3.0 g, 28.2 mmol) was loaded to the above reaction solution, followed by stirring at room temperature. 20 minutes later, the reaction was terminated by adding water. pH of the reaction solution was adjusted to 7 using 1 N HCl solution. Water layer was extracted three times with dichloromethane. Organic layer was washed with distilled water and brine three times. The organic layer was dried over anhydrous sodium sulfate and then dichloromethane was eliminated by vacuum distillation, followed by separation and purification to give pyruvoylaminoethane (620 mg, 19%).

Clear oil; ¹H NMR (CDCl₃) δ 3.34 (q, 2H), 2.47 (s, 3H), 1.26 (t, 3H)

### Manufacturing Example 2

### Preparation of Pyruvoylamininediethane

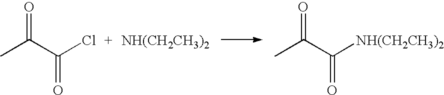

Diethaneamine (2.9 mL, 28.2 mmol) was dissolved in dichloromethane (40 mL). Triethylamine (4.0 mL, 28.2 mmol) was added thereto, followed by stirring for 5 minutes. Pyruvoyl chloride (3.0 g, 28.2 mmol) was loaded to the above reaction solution, followed by stirring at room temperature. 20 minutes later, the reaction was terminated by adding water. pH of the reaction solution was adjusted to 7 using 1 N HCl solution. Water layer was extracted three times with dichloromethane. Organic layer was washed with distilled water and brine three times. The organic layer was dried over anhydrous sodium sulfate and then dichloromethane was eliminated by vacuum distillation, followed by separation and purification to give pyruvoylamininediethane (650 mg, 16%).

Clear oil; ¹H NMR (CDCl₃) δ 3.44 (q, 2H), 3.32 (q, 2H), 2.42 (s, 3H), 1.21 (m, 6H)

### Manufacturing Example: 3

### Preparation of Pyruvoylthioethane

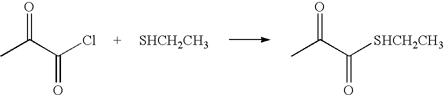

Ethanethiol (2.33 g, 37.6 mmol) was dissolved in dichloromethane (40 mL). The reaction solution was cooled down to 0° C., followed by stirring for 10 minutes. Pyruvoyl chloride (2.01 g, 18.8 mmol) was loaded thereto, followed by stirring for 10 minutes. Temperature of the reaction solution was slowly raised to room temperature, followed by drying over anhydrous sodium sulfate. The reaction solution was vacuum-filtered, vacuum-distilled, separated and purified to give pyruvoylthioethane (1.11 g, 45%).

Clear oil; ¹H NMR (CDCl₃) δ 2.94 (q, 2H), 2.42 (s, 3H), 1.30 (t, 3H)

### Comparative Example

### Evaluation of Cerebral Infarction Inhibiting Effect of Ethylpyruvate (EP)

To investigate neuroprotective effect of EP using a stroke animal model, local ischemic rat model was prepared according to the method of Longa (1989). Middle cerebral artery (MCA) of a rat was ligated for one hour using nylon suture to prepare a stroke animal model (middle cerebral artery occlusion, MCAO). To investigate therapeutic effect of EP on the ischemic brain, the animal was sacrificed after reperfusion and brain was isolated. The total brain was cut into 2 mm coronary sections by using metal brain template. The sections were stained immediately with 1% TTC (2,3,5-triphenyl tetrazolium chloride). Each section was stored in 4% paraformaldehyde solution at 37° C. for 15 minutes and cerebral ischemia regions thereof were measured and analyzed by using Quantity One program (Bio-Rad, Hercules, Calif., USA). MCAO occlusion was applied for one hour and EP was intravenously injected at different concentrations of 1, 5, 10, and 20 mg/kg.

### (1) Observation of Ischemia Regions in Brain Sections Over the Time and Concentration

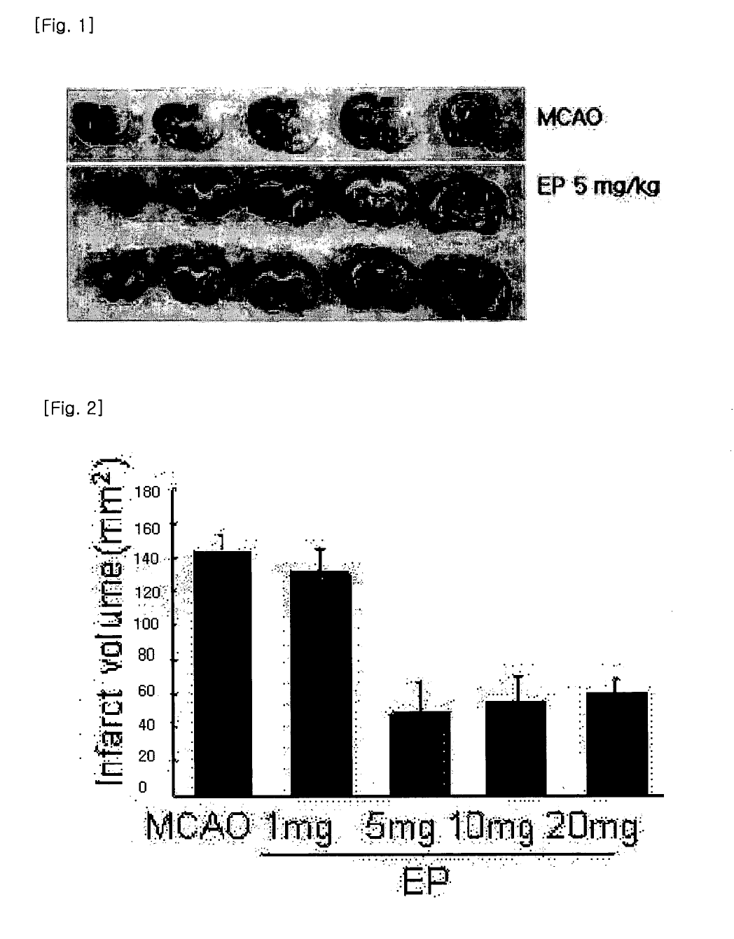

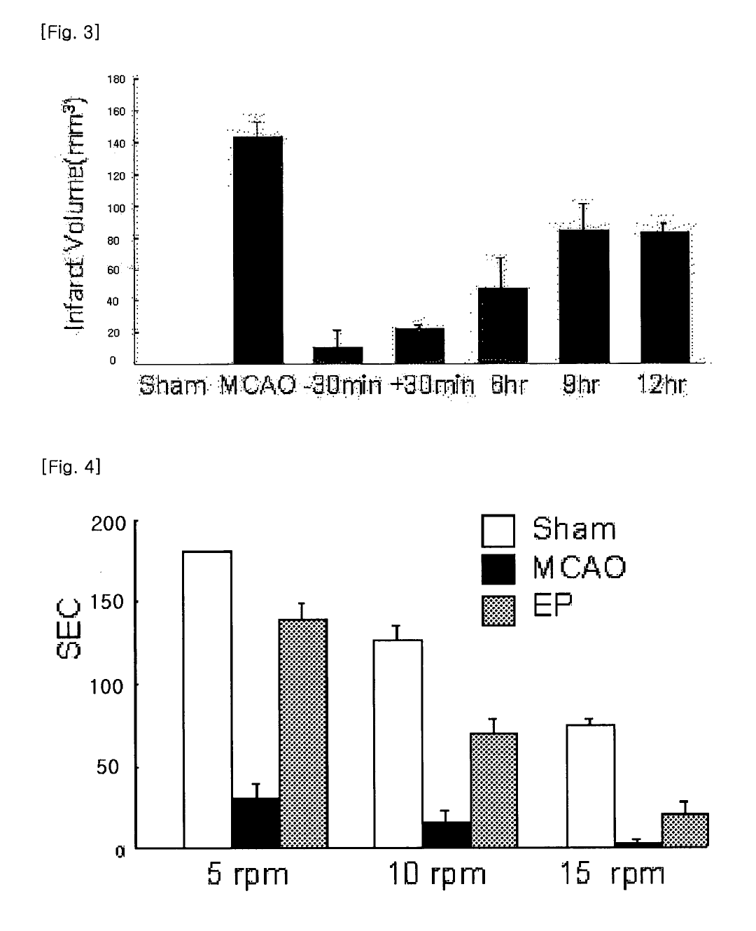

5 mg/kg of EP was administered to post-ischemic animal 6 hours after reperfusion, followed by TTC staining for evaluating infarct formation. As a result, infarcted regions shown as white were significantly decreased compared with that of the control group (FIG. 1). The MCAO model prepared by ligation for 1 hour was intravenously injected with EP at different concentrations of EP (1, 5, 10, and 20 mg/kg) 6 hours after reperfusion. The volume of ischemia region was not changed at the concentration of 1 mg/kg. However, the volume of ischemia region was reduced to 33.6%, 41.7% and 61.6% by the control animal respectively at the concentrations of 5, 10 and 20 mg/kg, respectively (FIG. 2). To determine effective time window for neuroprotection, EP was intravenously administered at the concentration of 5 mg/kg to the MCAO model 30 minutes before reperfusion and 30 minutes, 6, 9 and 12 hours after the reperfusion. As a result, the infarction volume was reduced to 9.0%, 17.5%, 34.0%, 58.7% and 58.0% respectively (FIG. 3). The above result confirmed the neuroprotective effect of EP on the brain with cerebral ischemia of the MCAO model.

### (2) Improvement of Motor Activity and Neurological Deficits

### (2-1) Rota-Rod Test

Motor skill regression and recognition deficit after brain damage by cerebral ischemia were reported previously (Yamamoto M. et al., Brain Res. 452: 323-328, 1988; Roger D. C. et al., Stroke 28: 2060-2065, 1997). Motor skill of a rat damaged by MCAO was investigated by measuring retention time on Rota-rod (Rota-rod test).

Retention time of each rat on Rota-rod at 5 rpm was measured for 24 hours after MCAO for 1 hour. Next, each rat was also forced to stay on Rota-rod operating at the speeds of 10 rpm and 15 rpm and retention time of each rat was measured at one hour interval. As a result, EP affords improvement of motor skill which were severely damaged by MCAO (FIG. 4). After 24 hours, there was no difference in behavior on Rota-rod operating at the speed of 5 rpm between normal rats and sham-operated rats. After one hour of MCAO/reperfusion, the behavior of rats with cerebral ischemia on Rota-rod was significantly regressed and in fact their stay on Rota-rod became notably shorter over the time. When 5 mg/kg of EP was intravenously injected 6 hours after MCAO/reperfusion, a rat spent significantly longer period on Rota-rod, suggesting that motor skill was recovered. The points herein were obtained from repeated experiments, which were performed at 1 hour interval at the speeds of 10 rpm and 15 rpm.

### (2-2) Evaluation of Neurologic Deficit

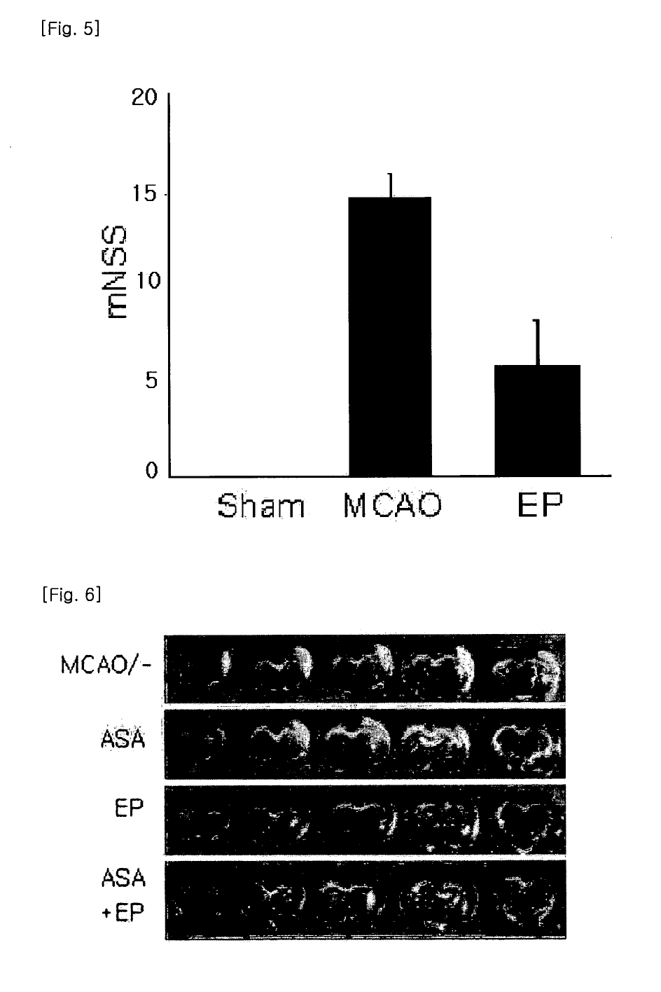

Neurologic deficit was investigated two days after MCAO/reperfusion. 5 different motor tests including modified static reflex test and hemiparesis test (Bederson J. B. et al., Stroke 17; 472-476) and forelimb placing test were hired to evaluate motor skill. Static reflex was evaluated by 4 point system (0: no observable deficit, 1: body bending and lifting an animal to the opposite tail, 2: rotating to the opposite direction, normal position while resting, 3: lying down in the opposite direction while resting, 4: no spontaneous movement) (Bederson J. B. et al., Stroke 17; 472-476). Forelimb placing was evaluated by the method described (0: immediate and complete placing, 1: incomplete and/or delayed placing, 2: no placing) (De Ryck M. et al., Stroke 20; 1388-1390). Points of static reflex and forelimb placing of each animal were summed. Motor skill deficit points (total 18 points) including above points were measured. As a result, EP was confirmed to suppress neurologic deficit after MCAO/reperfusion (FIG. 5).

### Example 1

### Cerebral Infarction Inhibiting Effect of Combination Therapy of Ethylpyruvate (EP) and Acetylsalicylic Acid

### (1) Measurement of Infarct Regions in Brain Sections Over the Time and Concentrations

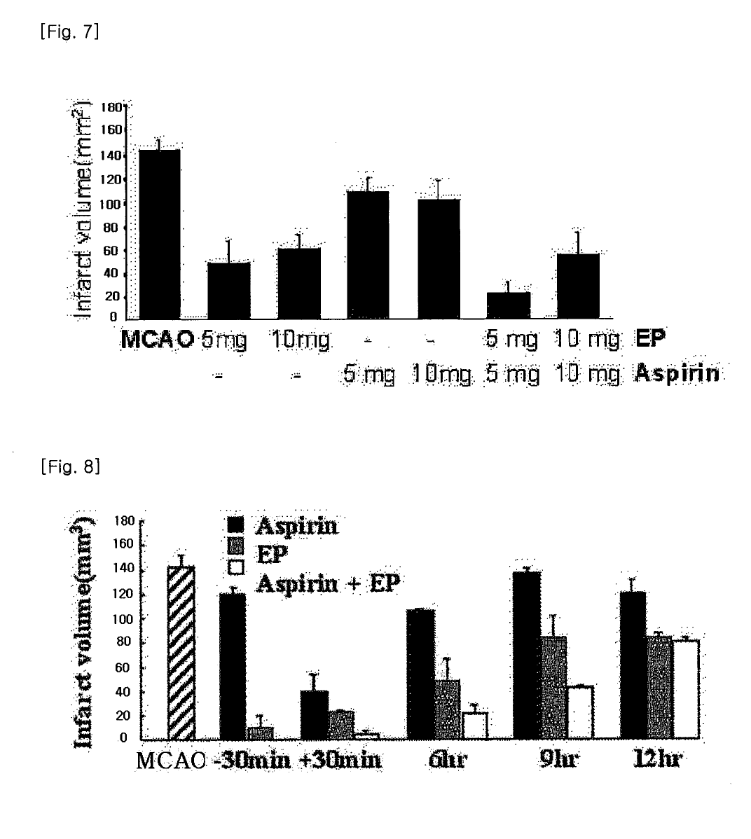

To investigate the effect of combination therapy of EP and acetylsalicylic acid, EP and acetylsalicylic acid were co-administered at the concentrations of 5, 10 and 20 mg/kg, respectively. In the case that EP and acetylsalicylic acid were co-administered at the concentration of 5 mg/kg each, infarction inhibiting effect was greater than when EP and acetylsalicylic acid were administered independently at the concentration of 10 mg/kg. At that time, infarction suppressing effect of combination therapy was the greatest at the concentration of 5 mg/kg. Even if the concentration was increased to 10 mg/kg each, the effect was not increased. The above result suggests that the effect of EP and acetylsalicylic acid is complementary (FIGS. 6 and 7).

To determine time window of constant effect of combination therapy of EP and acetylsalicylic acid, the effect of combination therapy was measured over hours. EP (5 mg/kg) and acetylsalicylic acid (5 mg/kg) were co-treated 30 minutes before 1 hour MCAO, 30 minutes, 6 hours, 9 hours and 12 hours after 1 hour MCAO. 24 hours after reperfusion, the volume of cerebral infarction region was measured by TTC staining. In the case of combination therapy 30 minutes before MCAO, the effect of EP was prominent and in the case of combination therapy 30 minutes after MCAO, the effect of EP was significantly increased. In the case of combination therapy 6 hours after MCAO, the effect of EP was consistently increased, which was also observed 9 hours after MCAO (FIG. 8).

### (2) Improvement of Motor Activity and Neurological Deficits

### (2-1) Rota-Rod Test

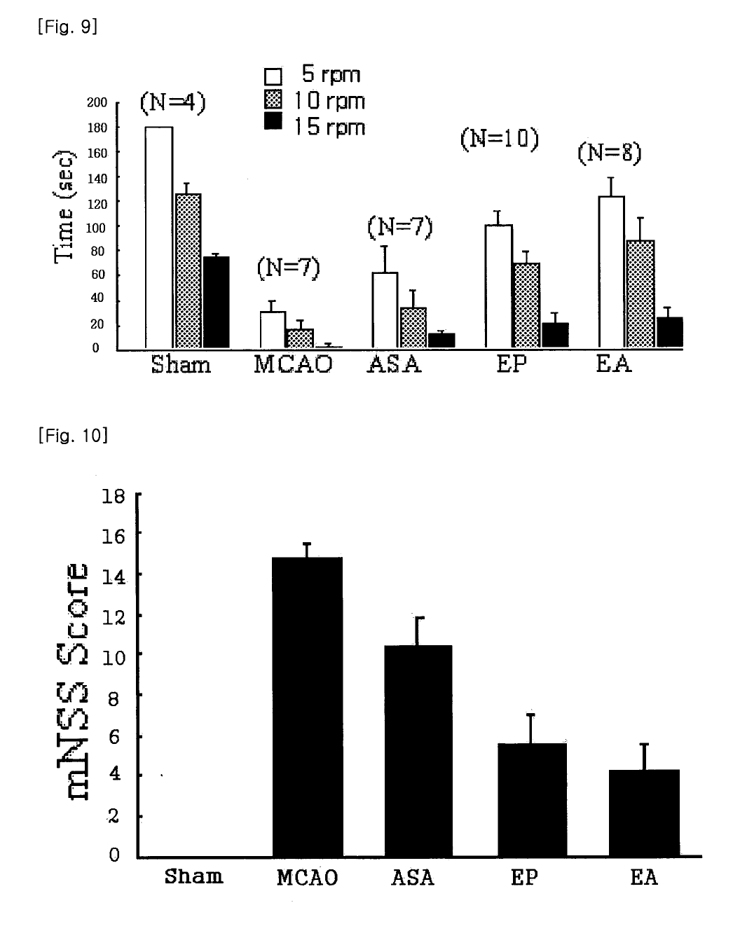

Rota-rod test was performed by the same manner as described in Comparative Example. After 6 hours from MCAO/reperfusion, 5 mg/kg of acetylsalicylic acid was intravenously injected. As a result, motor skill was recovered to 30% by normal motor skill. When 5 mg/kg of EP was intravenously injected after 6 hours from MCAO/reperfusion, motor skill was recovered to 70% by normal motor skill. In the meantime, combination therapy of EP and acetylsalicylic acid improved motor skill to 80% by normal motor skill (FIG. 9).

### (2-2) Evaluation of Neurologic Deficit

Neurologic deficit was evaluated by the method described in Comparative Example. When acetylsalicylic acid was administered alone 6 hours after MCAO/reperfusion, the recovery was very weak. When EP was administered alone, neuronal damage was improved 60%. Combination therapy of EP and acetylsalicylic acid significantly improved motor skill deficit to 72% (FIG. 10). The above results indicate that co-administration of EP and acetylsalicylic acid has significant suppressing effect on neurologic deficit after MCAO/reperfusion. That is, combination therapy has excellent cerebral infarction inhibiting effect and at the same time functional recovery effect, suggesting that functional recovery is also complementary.

### Example 2

### Cerebral infarction inhibiting effect of combination therapy of ethylpyruvate (EP) and chloro-2-hydroxy-5-(2,3,5,6-tetrafluoro-4-(trifluoromethyl)benzylamino)benzoate

### (1) Measurement of Infarct Regions in Brain Sections Over the Time

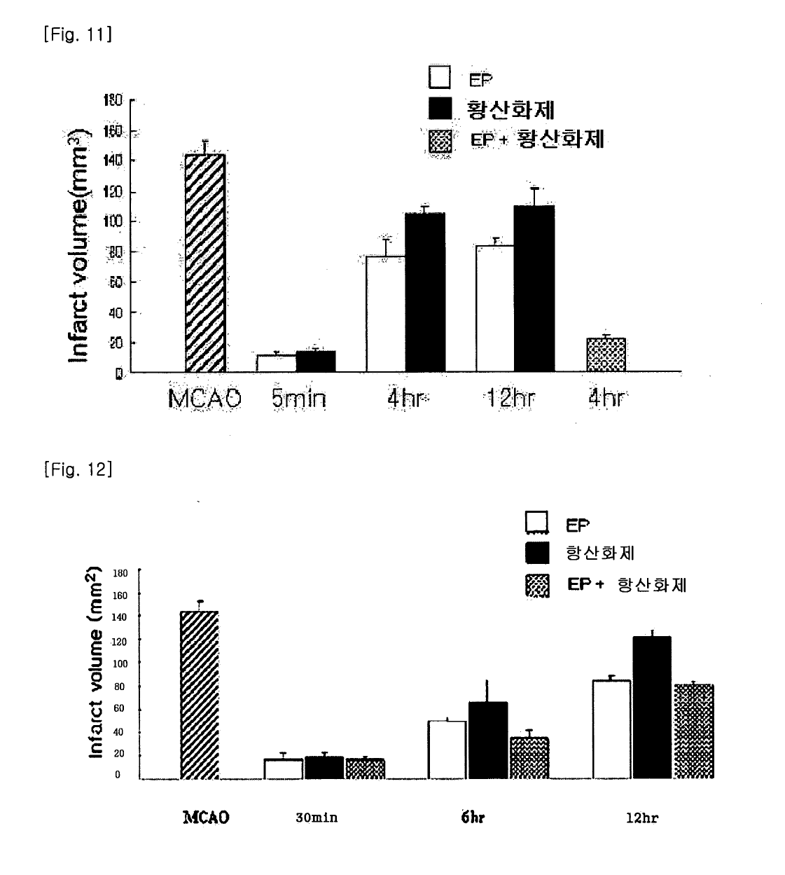

To investigate the effect of combination therapy of EP and chloro-2-hydroxy-5-(2,3,5,6-tetrafluoro-4-(trifluoromethyl)benzylamino)benzoate, each drug was administered by intravenous injection 5 minutes, 4 hours, 6 hours and 12 hours after reperfusion at the concentration of 5 mg/kg. When EP and chloro-2-hydroxy-5-(2,3,5,6-tetrafluoro-4-(trifluoromethyl)benzylamino)benzoate were independently administered 4 hours after reperfusion, cerebral infarction was inhibited by 48% and 30% respectively. In the case of co-administration of EP and chloro-2-hydroxy-5-(2,3,5,6-tetrafluoro-4-(trifluoromethyl)benzylamino)benzoate, cerebral infarction was inhibited up to 76%, indicating significant complementary effect (FIG. 11). Such complementary effect of combination therapy continued even after 6 hours from reperfusion (FIG. 12).

### (2) Improvement of Neurological Deficits

Neurologic deficit was evaluated by the method described in Comparative Example. Neuronal damage was effectively recovered as treatment was performed as early as possible.

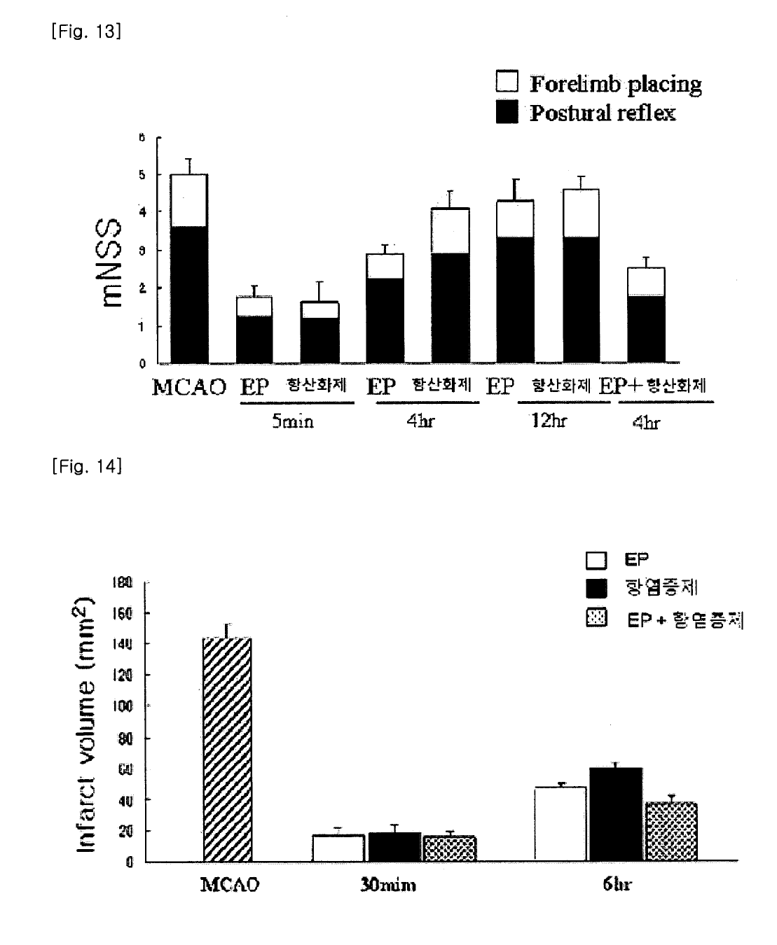

When chloro-2-hydroxy-5-(2,3,5,6-tetrafluoro-4-(trifluoromethyl)benzylamino)benzoate was administered alone after 4 hours from neuronal damage, the recovery was very weak. When EP was administered alone, neuronal damage was improved by 40%. When EP and chloro-2-hydroxy-5-(2,3,5,6-tetrafluoro-4-(trifluoromethyl)benzylamino)benzoate were co-administered, neurologic deficit was 50% recovered, suggesting that combination therapy brought 10% higher recovery effect than EP alone had (FIG. 13). That is, combination therapy of EP and chloro-2-hydroxy-5-(2,3,5,6-tetrafluoro-4-(trifluoromethyl)benzylamino)benzoate is more effective in reducing the volume of cerebral infarction and in improvement of motor skill, indicating the two drugs are complementary to each other.

### Example 3

### Cerebral Infarction Inhibiting Effect of Combination Therapy of Ethylpyruvate (EP) and chloro-2-hydroxy-5-(4-(trifluoromethyl)phenylethylamino) benzoate

### (1) Measurement of Ischemia Regions in Brain Sections Over the Time

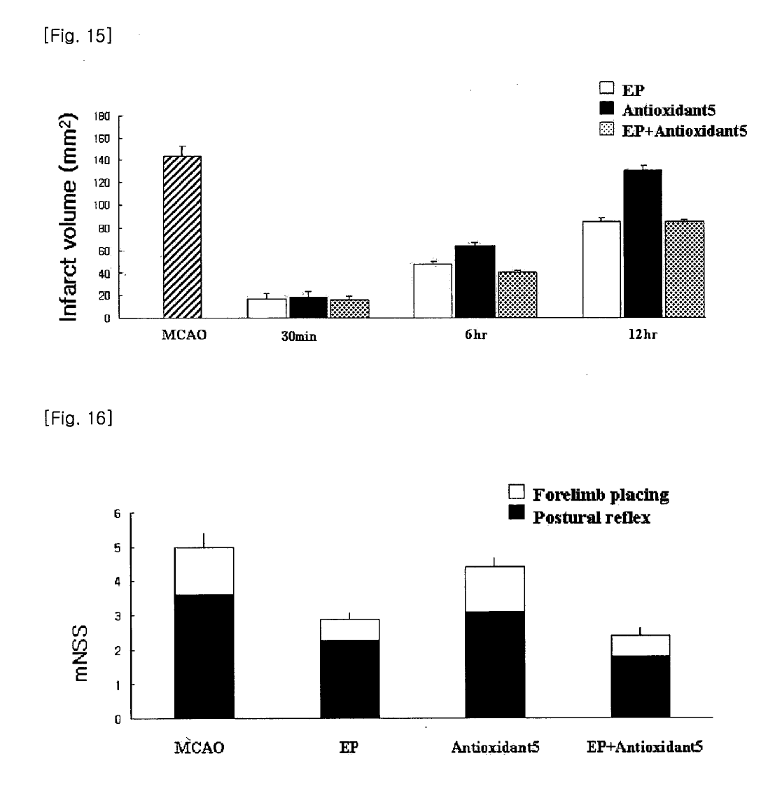

To investigate the effect of combination therapy of EP and chloro-2-hydroxy-5-(4-(trifluoromethyl)phenylethylamino)benzoate, each drug was administered by intravenous injection 30 minutes, 6 hours and 12 hours after reperfusion at the concentration of 5 mg/kg. When EP and chloro-2-hydroxy-5-(4-(trifluoromethyl)phenylethylamino)benzoate were co-administered 6 hours after reperfusion, infarction inhibiting effect was greater than single treatment at the concentration of 5 mg/kg each. Precisely, EP and chloro-2-hydroxy-5-(4-(trifluoromethyl)phenylethylamino)benzoate reduced the volume of cerebral infarction by 66% and 58% respectively, while combination therapy of the two drugs reduced the volume of cerebral infarction by 74%, suggesting that EP and chloro-2-hydroxy-5-(4-(trifluoromethyl)phenylethylamino)benzoate are complementary to each other (FIG. 14).

### Example 4

### Cerebral Infarction Inhibiting Effect of Combination Therapy of Ethylpyruvate (EP) and chloro-5-(2-(4-chlorophenoxy)ethylamino)-2-hydroxybenzoic Acid

### (1) Measurement of Ischemia Regions in Brain Sections Over the Time

To investigate the effect of combination therapy of EP and chloro-5-(2-(4-chlorophenoxy)ethylamino)-2-hydroxybenzoic acid, each drug was administered by intravenous injection 30 minutes, 6 hours and 12 hours after reperfusion at the concentration of 5 mg/kg. When EP and chloro-5-(2-(4-chlorophenoxy)ethylamino)-2-hydroxybenzoic acid were co-administered 6 hours after reperfusion, infarction inhibiting effect was greater than single treatment at the concentration of 5 mg/kg each. Precisely, EP and chloro-5-(2-(4-chlorophenoxy)ethylamino)-2-hydroxybenzoic acid reduced the volume of cerebral infarction by 66% and 55% respectively, while combination therapy of the two drugs reduced the volume of cerebral infarction by 72%, suggesting that EP and chloro-5-(2-(4-chlorophenoxy)ethylamino)-2-hydroxybenzoic acid are complementary to each other (FIG. 15).

### (2) Improvement of Neurological Deficits

Neurologic deficit was evaluated by the method described in Comparative Example. Neuronal damage was effectively recovered as treatment was performed as early as possible. When chloro-5-(2-(4-chlorophenoxy)ethylamino)-2-hydroxybenzoic acid was administered alone after 6 hours from neuronal damage, the recovery was very weak. When EP was administered alone, neuronal damage was improved 40%. When EP and chloro-5-(2-(4-chlorophenoxy)ethylamino)-2-hydroxybenzoic acid were co-administered, neurologic deficit was 45% recovered, suggesting that combination therapy brought 5% higher recovery effect than EP alone had (FIG. 16). That is, combination therapy of EP and chloro-5-(2-(4-chlorophenoxy)ethylamino)-2-hydroxybenzoic acid is more effective in reducing the volume of cerebral infarction and in improvement of motor skill, indicating the two drugs are complementary to each other.

### Example 5

### Cerebral Infarction Inhibiting Effect of Combination Therapy of Ethylpyruvate (EP) and chloro-5-(2-(2,4-dichlorophenoxy)ethylamino)-2-hydroxybenzoic Acid

### (1) Measurement of Ischemia Regions in Brain Sections Over the Time

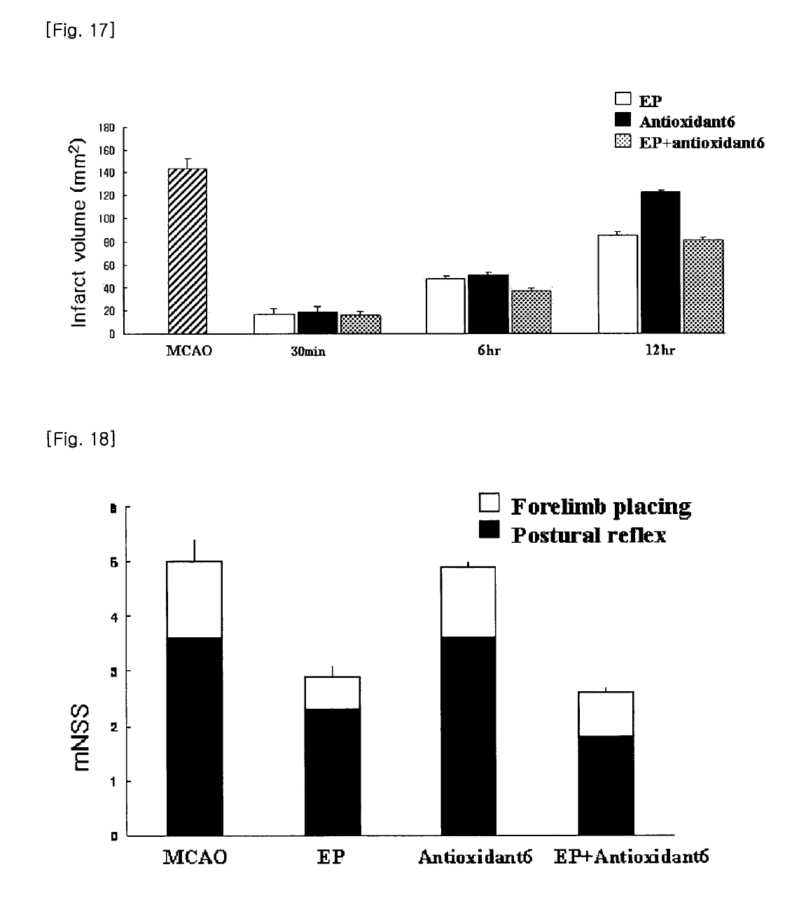

To investigate the effect of combination therapy of EP and chloro-5-(2-(2,4-dichlorophenoxy)ethylamino)-2-hydroxybenzoic acid, each drug was administered by intravenous injection 30 minutes, 6 hours and 12 hours after reperfusion at the concentration of 5 mg/kg. When EP and chloro-5-(2-(2,4-dichlorophenoxy)ethylamino)-2-hydroxybenzoic acid were co-administered 6 hours after reperfusion, infarction inhibiting effect was greater than that of single treatment at the concentration of 5 mg/kg each. Precisely, EP and chloro-5-(2-(2,4-dichlorophenoxy)ethylamino)-2-hydroxybenzoic acid reduced the volume of cerebral infarction by 66% and 61% respectively, while combination therapy of the two drugs reduced the volume of cerebral infarction by 74%, suggesting that EP and chloro-5-(2-(2,4-dichlorophenoxy)ethylamino)-2-hydroxybenzoic acid are complementary to each other (FIG. 17).

### (2) Improvement of Neurological Deficits

Neurologic deficit was evaluated by the method described in Comparative Example. Neuronal damage was effectively recovered as treatment was performed as early as possible. When chloro-5-(2-(2,4-dichlorophenoxy)ethylamino)-2-hydroxybenzoic acid was administered alone after 6 hours from neuronal damage, the recovery was very weak. When EP was administered alone, neuronal damage was improved 40%. When EP and chloro-5-(2-(2,4-dichlorophenoxy)ethylamino)-2-hydroxybenzoic acid were co-administered, neurologic deficit was 52% recovered, suggesting that combination therapy brought 12% higher recovery effect than EP alone had (FIG. 18). That is, combination therapy of EP and chloro-5-(2-(2,4-dichlorophenoxy)ethylamino)-2-hydroxybenzoic acid is more effective in reducing the volume of cerebral infarction and in improvement of motor skill, indicating the two drugs are complementary to each other (FIG. 18).

### Example 6

### Cerebral Infarction Inhibiting Effect of Combination Therapy of Ethylpyruvate (EP) and chloro-2-hydroxy-5-(2-(2,4,5-trichlorophenoxy)ethylamino)benzoic Acid

### (1) Measurement of Ischemia Regions in Brain Sections Over the Time

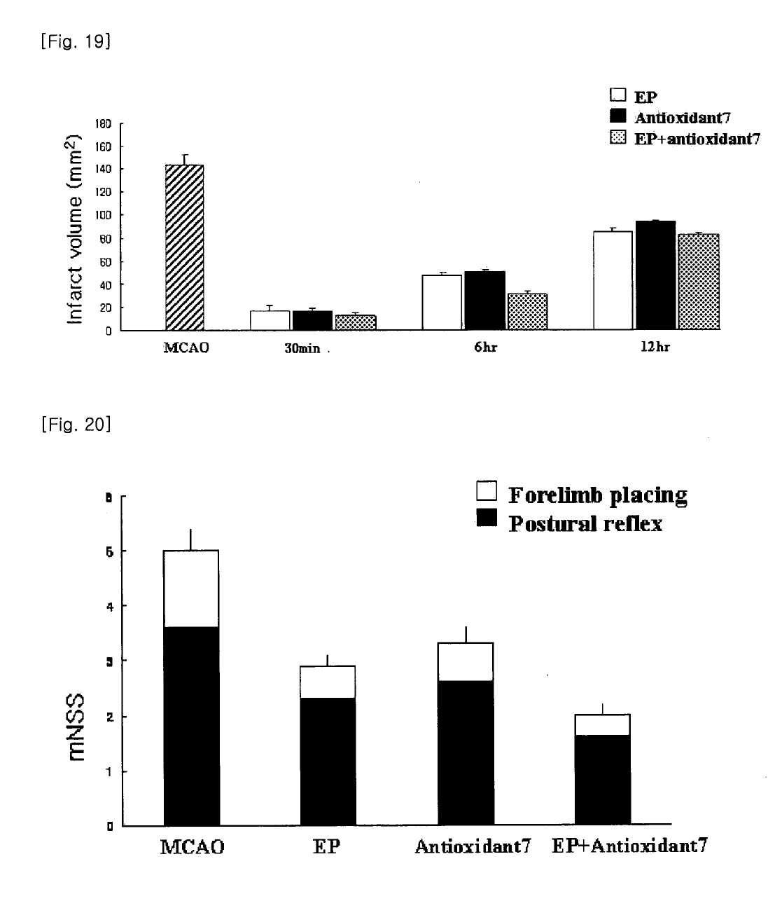

To investigate the effect of combination therapy of EP and chloro-2-hydroxy-5-(2-(2,4,5-trichlorophenoxy)ethylamino)benzoic acid, each drug was administered by intravenous injection 30 minutes, 6 hours and 12 hours after reperfusion at the concentration of 5 mg/kg. When EP and chloro-2-hydroxy-5-(2-(2,4,5-trichlorophenoxy)ethylamino)benzoic acid were co-administered 6 hours after reperfusion, infarction inhibiting effect was greater than single treatment at the concentration of 5 mg/kg each. Precisely, EP and chloro-2-hydroxy-5-(2-(2,4,5-trichlorophenoxy)ethylamino)benzoic acid reduced the volume of cerebral infarction by 66% and 64% respectively, while combination therapy of the two drugs reduced the volume of cerebral infarction by 78%, suggesting that EP and chloro-2-hydroxy-5-(2-(2,4,5-trichlorophenoxy)ethylamino)benzoic acid are complementary to each other (FIG. 19).

### (2) Improvement of Neurological Deficits

Neurologic deficit was evaluated by the method described in Comparative Example. Neuronal damage was effectively recovered as treatment was performed as early as possible. When chloro-2-hydroxy-5-(2-(2,4,5-trichlorophenoxy)ethylamino)benzoic acid was administered alone after 6 hours from neuronal damage, the recovery was very weak. When EP was administered alone, neuronal damage was improved by 40%. When EP and chloro-2-hydroxy-5-(2-(2,4,5-trichlorophenoxy)ethylamino)benzoic acid were co-administered, neurologic deficit was recovered by 55%, suggesting that combination therapy brought 15% higher recovery effect than EP alone had (FIG. 20). That is, combination therapy of EP and chloro-2-hydroxy-5-(2-(2,4,5-trichlorophenoxy)ethylamino)benzoic acid is more effective in reducing the volume of cerebral infarction and in improvement of motor skill, indicating the two drugs are complementary to each other.

### Example 7

### Cerebral Infarction Inhibiting Effect of Combination Therapy of Ethylpyruvate (EP) and chloro-2-hydroxy-5-(2-(4-methoxyphenoxy)ethylamino)benzoic Acid

### (1) Measurement of Ischemia Regions in Brain Sections Over the Time

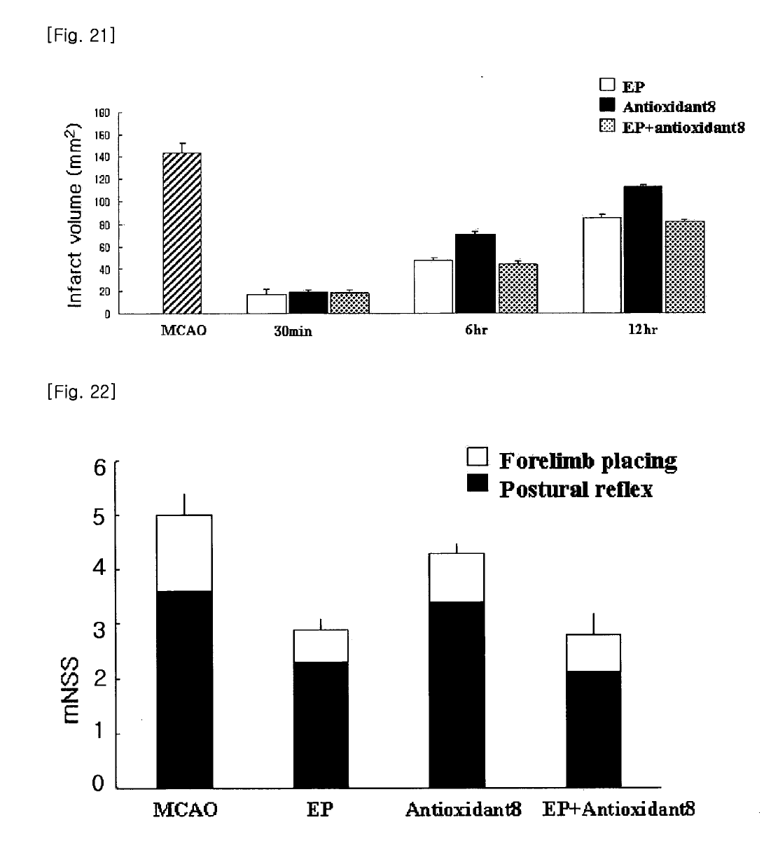

To investigate the effect of combination therapy of EP and chloro-2-hydroxy-5-(2-(4-methoxyphenoxy)ethylamino)benzoic acid, each drug was administered by intravenous injection 30 minutes, 6 hours and 12 hours after reperfusion at the concentration of 5 mg/kg. When EP and chloro-2-hydroxy-5-(2-(4-methoxyphenoxy)ethylamino)benzoic acid were co-administered 6 hours after reperfusion, infarction inhibiting effect was greater than single treatment at the concentration of 5 mg/kg each. Precisely, EP and chloro-2-hydroxy-5-(2-(4-methoxyphenoxy)ethylamino)benzoic acid reduced the volume of cerebral infarction by 66% and 53% respectively, while combination therapy of the two drugs reduced the volume of cerebral infarction by 69%, suggesting that EP and chloro-2-hydroxy-5-(2-(4-methoxyphenoxy)ethylamino)benzoic acid are complementary to each other (FIG. 21).

### (2) Improvement of Neurological Deficits

Neurologic deficit was evaluated by the method described in Comparative Example. Neuronal damage was effectively recovered as treatment was performed as early as possible. When chloro-2-hydroxy-5-(2-(4-methoxyphenoxy)ethylamino)benzoic acid was administered alone after 6 hours from neuronal damage, the recovery was very weak. When EP was administered alone, neuronal damage was improved by 40%. When EP and chloro-2-hydroxy-5-(2-(4-methoxyphenoxy)ethylamino)benzoic acid were co-administered, neurologic deficit was recovered by 43%, suggesting that combination therapy brought 3% higher recovery effect than EP alone had (FIG. 22). That is, combination therapy of EP and chloro-2-hydroxy-5-(2-(4-methoxyphenoxy)ethylamino)benzoic acid is more effective in reducing the volume of cerebral infarction and in improvement of motor skill, indicating the two drugs are complementary to each other.

### Example 8

### Cerebral Infarction Inhibiting Effect of Combination Therapy of Ethylpyruvate (EP) and Fluoxetine Chloride

### (1) Measurement of Ischemia Regions in Brain Sections Over the Time and Concentration

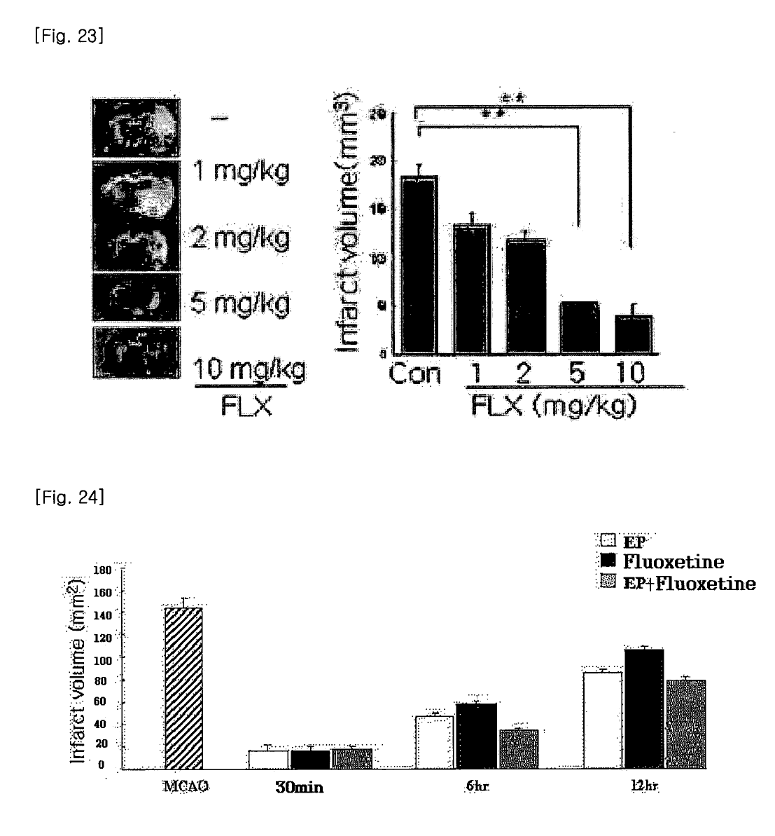

To investigate whether the cerebral infarction inhibiting effect of combination therapy of EP and fluoxetine chloride could be realized by the increase of concentrations of each drug, EP and fluoxetine chloride were treated at different concentrations of 1 mg/kg, 2 mg/kg, 5 mg/kg, 10 mg/kg and 20 mg/kg. After preparing one hour-local ischemia animal model, EP and fluoxetine chloride were administered by intravenous injection at different concentrations 6 hours after reperfusion, followed by measurement of the volume of cerebral infarction. Both drugs inhibited the size of cerebral infarction most effectively at the concentration of 5 mg/kg. Even if the concentration was increased to 10 mg/kg each, the effect was not increased (FIG. 23).

To investigate the effect of combination therapy of EP and fluoxetine chloride, each drug was administered by intravenous injection 30 minutes, 6 hours and 12 hours after reperfusion at the concentration of 5 mg/kg. When EP and fluoxetine chloride were co-administered 6 hours after reperfusion, infarction inhibiting effect was greater than that of single treatment at the concentration of 5 mg/kg each. Precisely, EP and fluoxetine chloride reduced the volume of cerebral infarction by 66% and 59% respectively, while combination therapy of the two drugs reduced the volume of cerebral infarction by 76%, suggesting that EP and fluoxetine chloride are complementary to each other (FIG. 24).

### (2) Improvement of Neurological Deficits

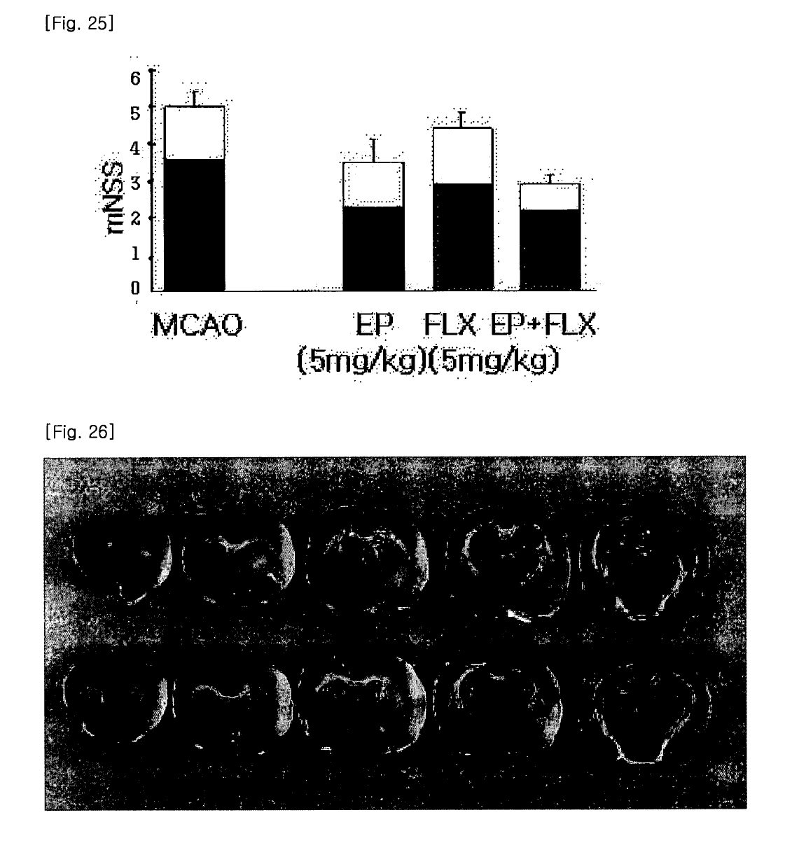

Neurologic deficit was evaluated by the method described in Comparative Example. Combination therapy of EP and fluoxetine chloride performed 6 hours after reperfusion demonstrated 10% higher neurologic deficit inhibiting effect than the single treatment of EP (FIG. 25). That is, combination therapy of EP and fluoxetine chloride is more effective in reducing the volume of cerebral infarction and in improvement of motor skill, indicating the two drugs are complementary to each other.

### Example 9

### Cerebral Infarction Inhibiting Effect of Combination Therapy of Pyruvoylamininediethane and 2-acetoxy-4-(trifluoromethyl)benzoic Acid (Trifusal)

### (1) Measurement of Ischemia Regions in Brain Sections Over the Time

To investigate the effect of combination therapy of pyruvoylamininediethane (Manufacturing Example 2) and 2-acetoxy-4-(trifluoromethyl)benzoic acid, each drug was administered by intravenous injection 30 minutes, 6 hours and 12 hours after reperfusion at the concentration of 2.5 mg/kg. When pyruvoylamininediethane and 2-acetoxy-4-(trifluoromethyl)benzoic acid were co-administered 6 hours after reperfusion, infarction inhibiting effect was greater than that of single treatment at the concentration of 2.5 mg/kg each. Precisely, pyruvoylamininediethane and 2-acetoxy-4-(trifluoromethyl)benzoic acid reduced the volume of cerebral infarction by 40% and 23% respectively, while combination therapy of the two drugs reduced the volume of cerebral infarction by 60%, suggesting that pyruvoylamininediethane and 2-acetoxy-4-(trifluoromethyl)benzoic acid are complementary to each other (FIG. 26).

### (2) Improvement of Neurological Deficits

Neurologic deficit was evaluated by the method described in Comparative Example. Combination therapy of pyruvoylamininediethane and 2-acetoxy-4-(trifluoromethyl)benzoic acid performed 6 hours after reperfusion demonstrated 30% higher neurologic deficit inhibiting effect than the single treatment of EP. That is, combination therapy of pyruvoylamininediethane and 2-acetoxy-4-(trifluoromethyl)benzoic acid is more effective in reducing the volume of cerebral infarction and in improvement of motor skill, indicating the two drugs are complementary to each other.

### Example 10

### Cerebral Infarction Inhibiting Effect of Combination Therapy of Pyruvoylamininediethane and 2-hydroxy-4-trifluoromethylbenzoic Acid (HTB)

### (1) Measurement of Ischemia Regions in Brain Sections Over the Time

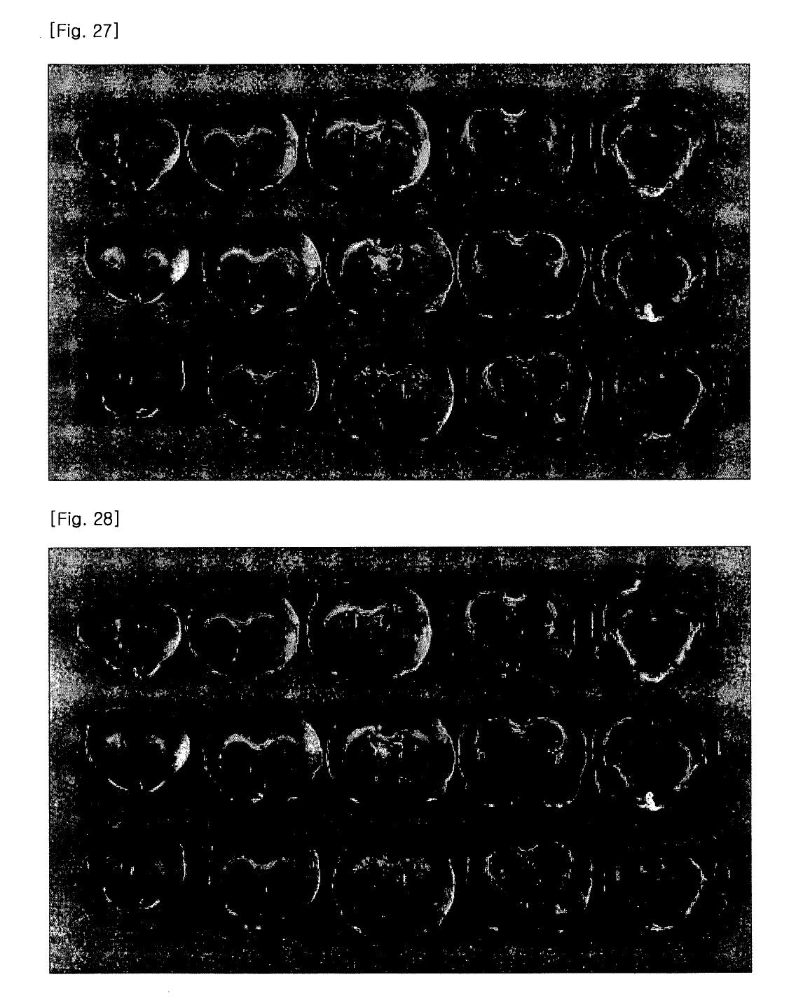

To investigate the effect of combination therapy of pyruvoylamininediethane (Manufacturing Example 2) and 2-hydroxy-4-trifluoromethylbenzoic acid, each drug was administered by intravenous injection 30 minutes, 6 hours and 12 hours after reperfusion at the concentration of 2.5 mg/kg. When pyruvoylamininediethane and 2-hydroxy-4-trifluoromethylbenzoic acid were co-administered 6 hours after reperfusion, infarction inhibiting effect was greater than that of single treatment at the concentration of 2.5 mg/kg each. Precisely, pyruvoylamininediethane and 2-hydroxy-4-trifluoromethylbenzoic acid reduced the volume of cerebral infarction by 31% and 30% respectively, while combination therapy of the two drugs reduced the volume of cerebral infarction by 42%, suggesting that pyruvoylamininediethane and 2-hydroxy-4-trifluoromethylbenzoic acid are complementary to each other (FIG. 27).

### (2) Improvement of Neurological Deficits

Neurologic deficit was evaluated by the method described in Comparative Example. Combination therapy of pyruvoylamininediethane and 2-hydroxy-4-trifluoromethylbenzoic acid performed 6 hours after reperfusion demonstrated 17% higher neurologic deficit inhibiting effect than the single treatment of EP. That is, combination therapy of pyruvoylamininediethane and 2-hydroxy-4-trifluoromethylbenzoic acid is more effective in reducing the volume of cerebral infarction and in improvement of motor skill, indicating the two drugs are complementary to each other.

### Example 11

### Cerebral Infarction Inhibiting Effect of Combination Therapy of Pyruvoylaminoethane and 2-acetoxy-4-(trifluoromethyl)benzoic Acid (Trifusal)

### (1) Measurement of Ischemia Regions in Brain Sections Over the Time

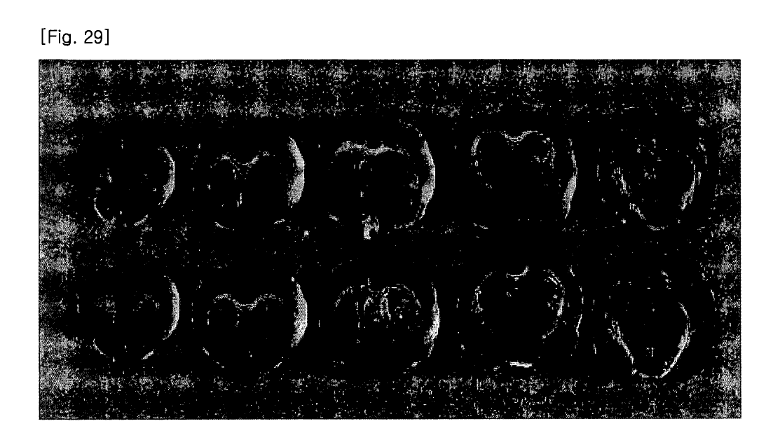

To investigate the effect of combination therapy of pyruvoylaminoethane (Manufacturing Example 1) and 2-acetoxy-4-(trifluoromethyl)benzoic acid, each drug was administered by intravenous injection 30 minutes, 6 hours and 12 hours after reperfusion at the concentration of 2.5 mg/kg. When pyruvoylaminoethane and 2-acetoxy-4-(trifluoromethyl)benzoic acid were co-administered 6 hours after reperfusion, infarction inhibiting effect was greater than that of single treatment at the concentration of 2.5 mg/kg each. Precisely, pyruvoylaminoethane and 2-acetoxy-4-(trifluoromethyl)benzoic acid reduced the volume of cerebral infarction by 20% and 23% respectively, while combination therapy of the two drugs reduced the volume of cerebral infarction by 54%, suggesting that pyruvoylaminoethane and 2-acetoxy-4-(trifluoromethyl)benzoic acid are complementary to each other (FIG. 28).

### (2) Improvement of Neurological Deficits

Neurologic deficit was evaluated by the method described in Comparative Example. Combination therapy of pyruvoylaminoethane and 2-acetoxy-4-(trifluoromethyl)benzoic acid performed 6 hours after reperfusion demonstrated 18% higher neurologic deficit inhibiting effect than the single treatment of EP. That is, combination therapy of pyruvoylaminoethane and 2-acetoxy-4-(trifluoromethyl)benzoic acid is more effective in reducing the volume of cerebral infarction and in improvement of motor skill, indicating the two drugs are complementary to each other.

### Example 12

### Cerebral Infarction Inhibiting Effect of Combination Therapy of Pyruvoylaminoethane and 2-hydroxy-4-trifluoromethylbenzoic Acid (HTB)

### (1) Measurement of Ischemia Regions in Brain Sections Over the Time

To investigate the effect of combination therapy of pyruvoylaminoethane (Manufacturing Example 1) and 2-hydroxy-4-trifluoromethylbenzoic acid, each drug was administered by intravenous injection 30 minutes, 6 hours and 12 hours after reperfusion at the concentration of 2.5 mg/kg. When pyruvoylaminoethane and 2-hydroxy-4-trifluoromethylbenzoic acid were co-administered 6 hours after reperfusion, infarction inhibiting effect was greater than that of single treatment at the concentration of 2.5 mg/kg each. Precisely, pyruvoylaminoethane and 2-hydroxy-4-trifluoromethylbenzoic acid reduced the volume of cerebral infarction by 25% and 30% respectively, while combination therapy of the two drugs reduced the volume of cerebral infarction by 49%, suggesting that pyruvoylaminoethane and 2-hydroxy-4-trifluoromethylbenzoic acid are complementary to each other (FIG. 29).

### (2) Improvement of Neurological Deficits

Neurologic deficit was evaluated by the method described in Comparative Example. Combination therapy of pyruvoylaminoethane and 2-hydroxy-4-trifluoromethylbenzoic acid performed 6 hours after reperfusion demonstrated 12% higher neurologic deficit inhibiting effect than the single treatment of EP. That is, combination therapy of pyruvoylaminoethane and 2-hydroxy-4-trifluoromethylbenzoic acid is more effective in reducing the volume of cerebral infarction and in improvement of motor skill, indicating the two drugs are complementary to each other.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A neuroprotective agent containing:
(1) pyruvoyl derivative represented by formula 1; and
(2) one or more antioxidants selected from the group consisting of acetylsalicylic acid derivative represented by formula 2,5-aminosalicylic acid derivative represented by formula 3, fluoxetine derivative represented by formula 4 and pharmaceutically acceptable salts thereof as active ingredients: 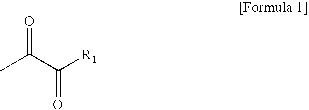
[Wherein, R₁ is (C1-C6) alkyl, (C6-C12) aryl, (C1-C6) alkoxy, (C6-C12) aryloxy, (C6-C12) ar (C1-C6) alkyl, (C6-C12) ar (C1-C6) or (C1-C6) alkylthio; R₂₁ and R₂₂ are independently H, (C1-C6) alkyl, (C6-C12) aryl, (C6-C12) ar (C1-C6) alkyl, (C6-C12) arylcarbonyl or (C1-C6) alkylcarbonyl; Alkyl, aryl, alkoxy, aryloxy, aralkyl, aralkyloxy, arylcarbonyl and alkylcarbonyl of R₁, R₂₁ and R₂₂ can be substituted with one or more substituents selected from the group consisting of halogen, halo (C1-C6) alkyl (in particular, CF₃), halo (C1-C6) alkyl (C6-C12) aryloxy and hydroxy.] 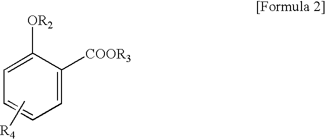
[Wherein, R₂ is H, (C1-C6) alkylcarbonyl or (C6-C12) arylcarbonyl; Arylcarbonyl of R₂ can be substituted with one or more substituents selected from the group consisting of (C1-C6) alkyl, hydroxy and halo (C1-C6) alkyl (in particular, CF₃); R₃ is H, (C1-C6) alkyl or (C6-C12) aryl; R₄ is H, (C1-C6) alkyl, halo (C1-C6) alkyl (in particular, CF₃), halogen, (C1-C6) alkoxy, hydroxy or nitro.] 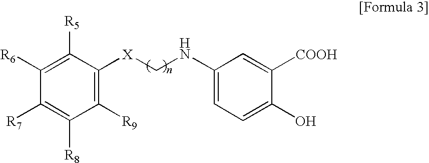
[Wherein, R₅-R₉ are independently H, (C1-C6) alkyl, halogen, hydroxy, (C1-C6) alkoxy, CF₃ or nitro; n is an integer of 0-4; X is CH₂, O, S or SO.] 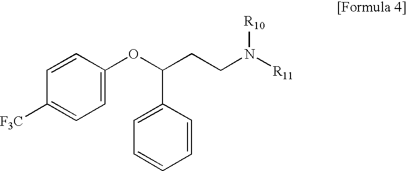
[Wherein, R₁₀ and R₁₁ are independently H, (C1-C6) alkyl or (C6-C12) aryl.]

2. The neuroprotective agent according to claim 1, wherein the agent is to prevent or treat cerebral ischemia, cerebral infarction, stroke, Huntington's disease, Lou Gehrig's disease or vascular dementia.

3. The neuroprotective agent according to claim 1 or claim 2, wherein the pyruvoyl derivative is selected from the group consisting of:
ethyl pyruvate (pyruvoyloxyethane);
propyl pyruvate;
isopropyl pyruvate;
n-butyl pyruvate;
sec-butyl pyruvate;
isobutyl pyruvate;
t-butyl pyruvate;
pentyl pyruvate;
hexyl pyruvate;
heptyl pyruvate;
octyl pyruvate;
phenyl pyruvate;
4-(4-trifluoromethylphenyloxy)phenyl pyruvate;
(4-trifluoromethyl)-2,3,5,6-tetrafluorophenyl pyruvate;
(4-trifluoromethyl)phenyl pyruvate;
(4-trifluoromethyl)benzyl pyruvate;
pyruvoylaminoethane;
pyruvoylaminomethane;
pyruvoylaminopropane;
pyruvoylaminobutane;
pyruvoylaminopentane;
pyruvoylaminobenzene;
pyruvoylamino(4-trifluoromethyl)benzene;
pyruvoylamino(4-trifluoromethylphenyl)methane;
pyruvoylamino(4-trifluoromethyl-2,3,5,6-tetrafluorophenyl)methane;
pyruvoylamino(4-trifluoromethyl-2,3,5,6-tetrafluoro)benzene;
pyruvoylaminocarbonyl(4-trifluoromethyl)benzene;
pyruvoylamininediethane;
pyruvoylamininedibutane;
pyruvoylamininedihexane;
pyruvoylamininedioctane;
pyruvoyl(4-trifluoromethyl-2-hydroxyphenylcarbonyl)(ethyl)amine;
pyruvoyl(4-trifluoromethyl-2-hydroxyphenyl)(ethyl)amine;
pyruvoyl(4-trifluoromethylphenyl)(ethyl)amine;
pyruvoylthioethane; and
pyruvoylpropane.

4. The neuroprotective agent according to claim 1 or claim 2, wherein the 5-aminosalicylic acid derivative is selected from the group consisting of:
2-hydroxy-5-(2,3,5,6-tetrafluoro-4-(trifluoromethyl)benzylamino)benzoic acid;
2-hydroxy-5-(4-(trifluoromethyl)phenylethylamino)benzoic acid;
5-(2-(4-chlorophenoxy)ethylamino)-2-hydroxybenzoic acid;
5-(2-(2,4-dichlorophenoxy)ethylamino)-2-hydroxybenzoic acid;
2-hydroxy-5-(2-(2,4,5-trichlorophenoxy)ethylamino)benzoic acid;
2-hydroxy-5-(2-phenylsulfonylethylamino)benzoic acid;
2-hydroxy-5-(2-(4-iodophenoxy)ethylamino)benzoic acid;
5-(2-(4-bromophenoxy)ethylamino)-2-hydroxybenzoic acid;
2-hydroxy-5-(3-phenoxypropylamino)benzoic acid;
5-(2-(2,6-dichloro-4-fluorophenoxy)ethylamino)-2-hydroxybenzoic acid;
5-(3-(4-fluorophenoxy)propylamino)-2-hydroxybenzoic acid;
5-(2-(2,6-dichloro-4-fluorophenoxy)ethylamino)-2-hydroxybenzoic acid;
5-(2-(4-chloro-2-methylphenoxy)ethylamino)-2-hydroxybenzoic acid;
2-hydroxy-5-(2-p-tolyloxyethylamino)benzoic acid;
2-hydroxy-5-(2-phenoxyethylamino) benzoic acid;
5-(2-(2,6-difluorophenoxy)ethylamino)-2-hydroxy benzoic acid; and
2-hydroxy-5-(2-(4-methoxyphenoxy)ethylamino)benzoic acid.

5. The neuroprotective agent according to claim 1 or claim 2, wherein the pharmaceutically acceptable salt is hydrochloride.

6. The neuroprotective agent according to claim 1 or claim 2, wherein the preferable dose of the pyruvoyl derivative is 1˜60 mg/kg and the preferable dose of the anti-oxidant is 1˜40 mg/kg.

7. The neuroprotective agent according to claim 1 or claim 2, wherein the pyruvoyl derivative and the anti-oxidant are included in a single or separated unit of formulation.

8. The neuroprotective agent according to claim 1 or claim 2, wherein the pyruvoyl derivative and the anti-oxidant are administered simultaneously or stepwise.

9. The neuroprotective agent according to claim 1 or claim 2, wherein the agent is administered intravenously or orally.

10. A pharmaceutical kit for neuroprotection containing:
(1) pyruvoyl derivative represented by formula 1; and
(2) one or more antioxidants selected from the group consisting of acetylsalicylic acid derivative represented by formula 2,5-aminosalicylic acid derivative represented by formula 3, fluoxetine derivative represented by formula 4 and pharmaceutically acceptable salts thereof as active ingredients. 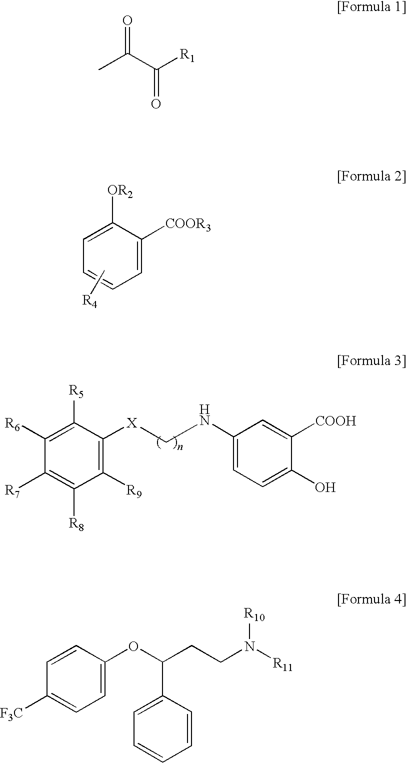
[In formulas 1-4, R₁-R₁₁, n and X are as defined in claim 1.]

11. A pharmaceutical package for neuroprotection containing:
(1) pyruvoyl derivative represented by formula 1; and
(2) one or more antioxidants selected from the group consisting of acetylsalicylic acid derivative represented by formula 2,5-aminosalicylic acid derivative represented by formula 3, fluoxetine derivative represented by formula 4 and pharmaceutically acceptable salts thereof as active ingredients. 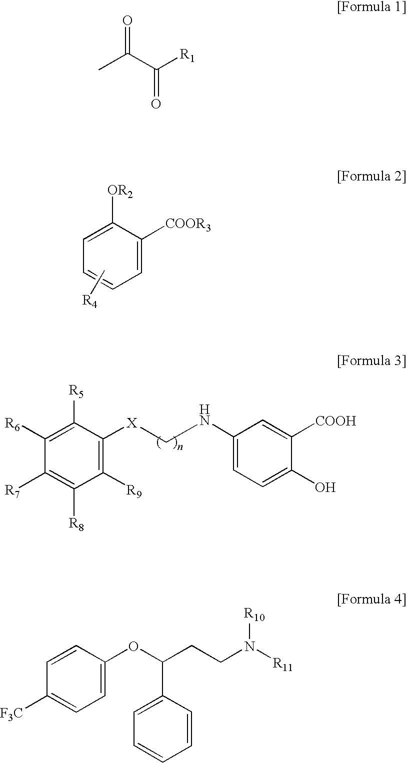
[In formulas 1-4, R₁-R₁₁, n and X are as defined in claim 1.]
